(19) United States
(12) Patent Application Publication
(10) Pub. No.: US 2004/0023922 A1
(43) Pub. Date: Feb. 5, 2004

(54) Lipids for modulating immune response

(76) Inventor: William Leslie Porter, Norfolk (GB)

Correspondence Address: William M Lee Jr, Lee Mann Smith McWilliams, Sweeney & Ohlson, P O Box 2786, Chicago, IL, 60690-2786, US

(21) Appl. No.: 10/311,023

(22) PCT Filed: Jun. 13, 2001
(86) PCT No.: PCT/GB01/02568

(30) Foreign Application Priority Data
Jun. 14, 2000 (GB) 0014437.8

(51) Int. Cl. A61K 31/739; A61K 31/685; A61K 31/225

(52) U.S. Cl. 514/54; 514/78; 514/547

(57) ABSTRACT

A product and the use thereof to stimulate and/or modulate immune response in animals and humans, comprising lipids having a glycerol backbone carrying at least one alkyl or acyl chain, wherein the lipid is phospholipid, glycolipid or neutral lipid, saturated or unsaturated, and the number of carbon atoms in the hydrocarbon tails lies between (10 and 22) inclusive, the lipids being defined as esters of glycerol (propane 1,2,3-triol) with fatty acids, also including 1,2-di-O-acylglycerol joined at oxygen (3) by a glycosidic linkage to a carbohydrate, also derivatives of glycerol in which one hydroxy group, commonly but not necessarly primary, is esterified with fatty acids, and execluding those organic compounds consisting of a trisaccharide repeating unit with oligosaccharide side chains and acyl or amine linked fatty acids.

### FIELD OF THE INVENTION

This invention relates generally to immune response modulation in man and animals and more especially to lipids and/or lipid-like preparations to cause or modulate immune response, both systemic and mucosal, preferably but not essentially effective by transmucosal administration. More particularly, the present invention concerns the use of lipid and lipid-like substances, of both bacterial and non-bacterial origin, administered to enhance or modulate the mucosal and systemic immune response to antigenic challenge, as a means of preventing and treating infectious and immune disease.

### BACKGROUND TO THE INVENTION

Control of infectious diseases in man and animals is a subject of major interest and research, since existing means of control have known deficiencies. Thus, antibiotics are continually rendered ineffective by the development of resistant strains of micro-organisms.

An opportunity therefore exists for the broad-spectrum enhancement of the natural immune response to infectious agents, as a means of treating and preventing a wide range of diseases. Since procedures involving parenteral injections can entail a risk of injection-site infections, particularly in third world situations, it would be preferable, although not exclusively so, that any such measure should be effective by the oral route.

It has been postulated that certain killed bacteria, and lipid extracts thereof incorporated in liposomes, given orally to animals, can enhance immune response to certain protein antigens. Antigenicity of certain bacterial lipids, given parenterally, is known, and an ability to cause immunomodulation has been reported for a few lipid-like chemical structures, given parenterally.

Enhancement of growth rate in young animals and the reduction of incidence of diarrhoea in animals orally receiving killed bacterial preparations, has also been reported, and it has been reported that enhancement of growth rate is achievable in animals receiving lipid-containing extracts of such bacterial preparations adsorbed onto non-lipid particulate matter, as well as in animals receiving similarly administered preparations of whole killed bacterial. It has also been shown that a chloroform/methanol-derived lipid extract of bacteria, given by mouth in liposomes, can enhance the cellular and antibody response to subsequent immune challenge.

It is also known that protein antigenic substances gain access from the gut to the reticulo-endothelial system via lymphoid tissue in the wall of the intestine, for instance Peyer's patches. Further it is known that such materials gain access more readily if they are incorporated in liposomes, bilosomes or similar lipid-containing vesicles or in beads formed by, for instance, polyethylene glycol.

In general, there is little knowledge of the identity of lipids which may modulate immune response in animals and humans, or the conditions necessary to render administration of lipids effective for the purpose, especially in the case or oral administration.

It is now known that there may be theoretical advantages in antigens and adjuvants gaining access to the immune system via mucosal surfaces, rather than by parenteral administration, in that access to the immune system may be facilitated.

Also, access via the mucosa, as by the gastro-enteric route, gives the possibility of modulating mucosal, as well as systemic, immune response.

A principal aim of the invention is to facilitate access to the body's immune system of certain bacterial lipids, which may be derived from non-pathogenic bacteria and/or certain non-bacterial lipids, preferably although not exclusively administered transmucosally, particularly orally, whereby to stimulate immunity and/or to modulate the immune response to antigenic stimulus. A means is also shown whereby the immune response to antigens may be suppressed, where it may be desirable to do so, as in the prevention and treatment of immune disease.

It is shown that lipids having particular physio-chemical characteristics are best suited for the purposes of this invention.

Principal Features of the Invention

According to one aspect of the invention there is provided a product to stimulate and/or modulate immune response in animals and humans, comprising lipids having a glycerol backbone carrying at least one alkyl or acyl chain, wherein the lipid is phospholipid, glycolipid or neutral lipid, saturated or unsaturated, and the number of carbon atoms in the hydrocarbon tails lies between 10 and 22 inclusive.

For the purpose of this application, lipids are defined as esters of glycerol (propane 1,2,3-triol) with fatty acids, also including 1,2-di-O-acylglycerol joined at oxygen 3 by a glycosidic linkage to a carbohydrate, also derivatives of glycerol in which one hydroxy group, commonly but not necessarily primary, is esterified with fatty acids, thus excluding those organic compounds consisting of a trisaccharide repeating unit with oligosaccharide side chains and acyl or amine linked fatty acids.

The complete product may comprise any one or a mixture of one or more of the aforesaid phospho- neutral or glyco lipids, which may be synthesised, produced by microbial fermentation, or may be contained in or derived from natural materials such as animal or vegetable oils from which they may or may not be extracted or concentrated as by distillation, fractionation, phoresis or other known processes.

The lipids are as specified in a) to i) below, given to man or animals in non-particulate or particulate form as specified below, by parenteral or transmucosal administration, for the purpose of modulating the immune response to antigens.

Specific Features of Lipids Employed in the Invention, Having Surprising Effects Upon Immune Response to Antigens

a) There should be one or more fatty acid chains, preferably two or more.

b) The chain length should be 10 to 22 carbon atoms, preferably 12 to 20 and ideally 16 or even 18 to 20.

c) Where there are two or more chains, the chains may be of equal length or, preferably, of unequal length.

d) Where a lipid shows optical isomerism, the d or l isomer may be employed, but preferably the d isomer.

e) Where a lipid has a head group, the head should preferably have a mass of at least 269 Atomic Mass Units (AMU) and a volume of at least 167 cm⁻³mol⁻¹.

f) Where a lipid has a head group, it may have a combined partial electrical charge of −1.015 eV or more strongly negative, but preferably of −1.044 eV or more strongly negative.

g) Where the head group has a charge, it may be in a single centre, but preferably should be in multiple centres.

h) The head group charge to volume ratio may should preferably be −0.004 eV/cm⁻³mol⁻¹ or more strongly negative, and ideally −0.006 eV/cm⁻³mol⁻¹ or more strongly negative.

i) Lipids may be saturated or unsaturated.

Whilst it might be postulated that lipids conforming to one or more of the above physico-chemical criteria might be particularly useful in facilitating linkage between antigens and receptor mechanisms of the immune system, the invention is by no means confined to this theory.

Further Features

Lipids may be administered trans-mucosally, or they may be administered parenterally. When given trans-mucosally, especially orally, for the purpose of enhancing immune response, it is desirable though not essential, that the lipids be given in particulate form. In the case of bacterial lipids, this may be achieved if the lipids are not extracted from the bacteria, the bacteria being subjected to a degradation step to render lipid components of the bacterial cells available to receptive areas of the gut wall or other mucosal surface, the degradation step being controlled so that the particle size of the lipid-bearing components, separately or in association with other materials, lies within predetermined limits. The degradation step may be by use of an enzyme, by autolysis, by heat treatment, such as boiling, autoclaving, irradiation (e.g. gamma irradiation) or microwaving, or by chemical treatment.

That the particle size is within the predetermined limits may be established by known methods, e.g. microscopic examination or by sizing techniques employing beams of radiation such as laser beams, and by readily available sizing instruments such as the Malvern Mastersizer.

Particle size of lipid-containing degraded or partially degraded bacteria may be at least partially controlled by known centrifugation techniques and further controlled by standard filtration procedures, so that particles of sizes outside the predetermined limits are largely or completely excluded. Particles of differing weights or densities may also be separated by centrifugation, for the same purpose.

The predetermined limits on lipid containing degraded or partially degraded bacteria particle size may be 0.1 microns to 100 microns and more specifically 0.2 to 25 microns. In particular 0.2 microns up to 15 microns or even 0.3 up to only 10 microns is preferred and ideally 0.4 to 8 microns or 0.5 to 5 microns.

Alternatively, lipids as specified in a) to i) above, consisting of or obtained from natural materials by means such as extraction with solvents such as chloroform and methanol, or alternatively of synthetic origin, may be rendered into a particulate form by incorporation into or onto liposomes or bilosomes or other appropriately sized particulate matter, such as silica, titanium dioxide, or kaolin particles, or other such material capable of absorbing or adsorbing lipids.

This may be achieved, for instance, by rendering the lipid or lipids into a liquid of low viscosity, as by heating, dissolving in solvents such as chloroform or methanol, or by forming into an aqueous preparation by means of an emulsifying agent, to form a emulsion or colloid.

The particles to be coated are then dispersed in the liquid by stirring, shaking vibration, sonication or other known means of dispersion. The liquid can then be removed to leave a coating of the lipid or lipid-like substance on the particle surfaces. This may be achieved by, for instance, evaporation of solvents or aqueous medium, centrifugation (batch or continuous flow) or filtration, preferably cross-flow filtration. When the lipid can be shown, for instance by microscopic examination, to be applied to the particles, a carrier may be added in suitable volume to provide a convenient dosage form.

It is preferable, though not essential that, where the lipid or lipids, (particularly if single chain, neutral or unsaturated) are administered in liposomes, bilosomes or other vesicular form, or as a suspension, protein or peptide antigens should not be directly mixed or combined with the lipid, or combined within vesicles. However, such antigens may be given concurrently with the lipid or lipids, in the same formulated dose, if not so directly combined. Alternatively, they may be given by separate administration, as in modulation of the immune response to antigenic challenge resulting from infection or other cause.

The lipid bacterial extracts or lipid-containing degraded bacteria or non-bacterial lipid, whether or not incorporated into liposomes, bilosomes or attached to suitably sized particles, may be incorporated into particulate formulations of wet or dry powder or granular mixes. Alternatively, they may be incorporated into liquids such as water, oil, milk or any beverage. The degraded bacteria or lipid, particulate or non-particulate, may also be incorporated as suspension in any of the aforementioned liquids. Powder formulation may also be given in tablets, capsules or paste, or may be incorporated into foods.

The lipid bacterial extracts or lipid-containing partially degraded bacteria may also be mixed with oil, which may be dispersed in water or other suitable liquid with or without the aid of emulsifying agents such as Crillet 4, forming an emulsion in which the bacterial extracts or degraded bacterial product is contained within or bound to the dispersed phase.

In the case of carrier particles, these should preferably have a size range of 0.1 to 100 microns, preferably 0.1 to 25 microns and most preferably 0.2 to 15 or even 0.3 to 10 microns, and ideally 0.4 to 8 microns or 0.5 to 5 microns.

One feature of the invention thus provides a preparation of lipids, as specified in a) to i) above, which may be administered parenterally or trans-mucosally in particulate form, with particles of dimensions within the above stated limits, or in non-particulate form.

In another feature of the invention, the lipids as specified above may be phospholipids, glycolipids or neutral lipids, saturated or unsaturated, with or without branching in their fatty acid chains, within the categories of:—

Phosphatidic acids

Phosphatidylethanolamines

Phosphatidylserines

Phosphatidylinositols

Phosphatidylcholines

Cardiolipins

Cerebrosides

Ceramides

Sphingosines

Sphingomylins

Triglycerides

1,2-Diglycerides

1,3-Diglycerides

Monoglycerides

and their isomers

More particularly, they may include:—

Phosphatidylcholine, dioleoyl

Trilinolenin

L-α-Phosphatidicacid, dimyristoyl

L-α-Phosphatidicacid, diheptadecanoyl

DL-α-Phosphatidylcholine, dimyristoyl

L-α-Phosphatidylcholine, dipentadecanoyl

L-α-Phosphatidylcholine, diheptadecanoyl

DL-α-Phosphatidylcholine, distearoyl

L-α-Phosphatidylethanolamine, dimyristoyl

DL-α-Phosphatidylethanolamine, dipalmitoyl

1,2-Distearoyl-sn-glycero-3-phosphoethanolamine

L-α-Phosphatidylethanolamine, diheptadecanoyl

DL-α-Phosphatidyl-L-serine, dipalmitoyl

The above are referred to as Group 1 Lipids, in Example 4, below.

N-Palmitoyl-d-sphigosine

N-Palmitoyl digydroglucocerebroside

Trymyristin

Tripentadecanoin

Tripalmitin

1,2 Diplamitoyl-3-myristoyl-rac-glycerol

1,2 Distearloyl-3-myristoyl-rac-glycerol

1,2 Distearoyl-3-palmitoyl-rac-glycerol

rac-1,2-Dimyristoyl-3-palmitoylglycerol

The above are referred to as Group 2 Lipids, in Example 4, below.

According to another aspect of the invention, there is provided the use of a lipid or lipids, as specified above, for promoting and/or modifying immune response in man and animals.

Such lipids may be given trans-mucosally, in particulate form as specified above, or they may be given parenterally, in particulate or non-particulate form. Trans-mucosal administration may be, for instance, by the oral route, by buccal or sub-lingual placement or spray, or by intranasal spray.

They may be given together with the antigen or antigens, as an adjuvant, or they may be given separately, especially prior to the antigenic challenge, in order enhance the immune response.

For some purposes of immune modulation, such as where the intention is to suppress rather than enhance the immune response to antigenic stimulus, for instance, in the control of immune diseases, the lipid materials may be given by a trans-mucosal route, but in non-particulate form. For these purposes, they may be administered dispersed in liquid emulsions, colloids, suspensions, solutions or other liquid dispersions or in gels, pastes or in dispersible in solid forms incorporating e.g. starch or cellulose, or may be administered in aerosol sprays.

The immunologically active material may be formed into liposomes by known techniques, or mixed with oily or viscous preparations to form liposomes, or may be otherwise incorporated into or onto carrier particles of appropriately sized particulate material, such as colloidal silica, silicilic acid, kaolin particles or other such material capable of absorbing or adsorbing lipids. Such particles may be incorporated into powder, tablets, capsules, food, liquids or beverages. The sizes of such liposomes or carrier particles incorporating the immunologically active material may have a size range of 0.1 to 100 microns, preferably 0.2 to 25 microns, most preferably 0.2 to 15 or even 0.3 to 10 microns, and ideally 0.4 to 8 microns or 0.5 to 5 microns.

Verification examples are now described.

### EXAMPLE 1

Comparison is made between two tests of immune globulin production in mice, following oral administration of a test antigen, where the mice received daily doses of partially autolysed and autoclaved Bacillus subtilis.

In test A, Bacillus subtilis was grown in a clear liquid medium containing yeast extract, sucrose, magnesium and other soluble nutrients, according to well-established procedures. The bacterial cells were separated from the culture medium by centrifugation and allowed to stand for 24 hours at room temperature. The cells were examined grossly and microscopically at intervals of about 6 hours. After this period, and before any visible physical breakdown of the bacteria was observed, the cells were autoclaved at 115 degrees C. for 20 minutes. Microscopically, the cells all remained substantially intact after autoclaving, with approximate dimensions of 0.5 to 1×1 to 5 microns.

The killed bacterial slurry was diluted in water and administered by gastric lavage, daily for 7 days, to 5 mice, at a daily dose rate equivalent to 400 micrograms of bacterial dry matter.

A similar group of 5 mice serving as a control group received daily oral doses of physiological saline.

Both groups were then antigenically challenged with keyhole limpet haemocyanin with cholera toxin. Both groups were then assayed for immune globulins.

The IgA response in the group receiving Bacillus subtilis preparation exceeded that in the control group by a factor of 9.

In test B, the above procedure was repeated, except that the Bacillus subtilis preparation was allowed to stand at room temperature for a period of 72 hours, so that autolytic changes progressed to a stage of microscopically visible breakdown of the bacterial cells. After the autoclaving, microscopy revealed that the bacterial preparation had broken down to a fine silt, of maximum particle size of 0.2 microns.

The antigenic challenge and subsequent assay of immune globulins was then carried out as in test A.

In test B, IgA response to the bacterial preparation was found to exceed that in the control group by a much reduced factor of 2.

Example 1 thus clearly demonstrates the beneficial effect of a killed bacterial preparation given orally as particles of 0.5 to 5 microns, compared to a similarly administered preparation of sub 0.2 micron particles.

### EXAMPLE 2

A comparison is made between two separate tests. In test C, chickens of from 1 to 21 days of age received food supplemented with a 2:1 methanol/chloroform extract of Bacillus subtilis, at the rate of the extract obtained from 100 mg of Bacillus subtilis dried biomass per kg of feed.

The methanol/chloroform extract, substantially lipid in nature, was applied to a dusty and finely granular preparation of expanded mica containing a high proportion of particles of approximately 0.2 to 100 microns, before being incorporated into the feed. Growth rate of treated chickens exceeded that of controls by 14.1 percent.

In test D, mice received a daily dose of a methanol/chloroform lipid extract of Bacillus subtilis administered by gastric lavage. The daily dose was the extract obtained from approximately 400 mg of dried biomass. Compared to controls, no weight gain enhancement, or enhancement of immune response, was detected.

Example 2 clearly demonstrates the beneficial effect of using an orally administered bacterial preparation or extract in particulate form as compared to a non-particulate form.

### EXAMPLE 3

Bacillus subtilis cells, cultured and subjected to partial autolysis, as in Example 1, test A, were agitated for 48 hours in a mixture of 2:1 chloroform and methanol. Following centrifugation, the supernatant was concentrated by evaporation to yield a lipid extract.

The extract was combined with a liposome in a buffered saline solution to give dispersed particles of lipid/liposome of approximately 1 to 3 microns in diameter. This was administered to 5 mice by daily gastric lavage for 5 days, in a total daily dose per mouse of 0.2 ml, giving a total dose per mouse of 0.59 mg of extracted lipid. Five control mice received the same liposome preparation, but without the bacterial lipid extract, suspended in identical buffered saline and also administered at 0.2 ml per mouse per day.

On days 7 and 15, all the mice were immunised by gastric lavage with keyhole limpet haemocyanin (5 mg) in 200 mcl of physiological saline containing cholera toxin (10 μg). On day 21, all the mice were bled and mesenteric lymph nodes removed.

Tests by ELISA of specific antibodies in serum showed levels of IgA in the lipid extract treated mice to exceed the control mice by a factor of 8.

Cell cultures of mesenteric lymph nodes showed T cell proliferation in nodes of treated animals in response to keyhole haemocyanin to exceed that in control animals by factors of 35 and 16 following 3 and 5 days of culture.

The results therefore showed marked modulation of immune response through oral pre-treatment with bacterial lipid extracts given in particles of approximately 1 to 3 microns.

It should be noted that the method of extraction employed in Example 3 was specifically selected to extract only lipids from the bacteria. Lipopolysaccharides, such as those known as Lipid A, are not extractable by the method employed.

### EXAMPLE 4

Examples of a product in accordance with the invention comprise mixtures of approximately the same amounts of all the lipids comprising the aforesaid Group 1, and also of the aforesaid Group 2. All these lipids can be synthetically produced.

A further example tested was an extract of chloroform and methanol, (2:1 C/M) obtained from Bacillus subtilis cells, contained in liposomes formed according to the method of Bangham and Horne (1964).

The same C/M extract of B. subtilis was also tested by oral administration of the extract applied to 1.5 micron particles of silicon dioxide. Lipids of Groups 1 and 2 were also administered orally in this way.

Test Procedure

The aforesaid exemplary products have been tested on four groups of mice, orally treated with the products, and compared with two control groups. In all groups, measurements were made of systemic and mucosal immune responses to protein antigen keyhole limpet hemocyanin-KLH) following treatment with the exemplary products.

Materials and Methods

Mice

Female Balb/c mice, 6-8 weeks old and weighing 20-22 gms were sued and maintained on a normal mouse diet throughout the study.

Sample Preparation

Samples were prepared for group treatments, as follows:

A) Physiological buffered saline (PBS) (Control treatment).

B) C/M extract of Bacillus subtilis, prepared in liposomes according to the method of Bangham and Horne (1964).

C) C/M extract of Bacillus subtilis, applied to 1.5 micron particles of silicon dioxide.

D) Non-bacterial phospholipids (Group 1 above) applied to silicon dioxide particles as in C).

E) Non-bacterial neutral lipids and glycolipids (Group 2 above) applied to silicon dioxide particles as in C).

F) Liposomes prepared according to the method of Bangham and Horne (1964) with no contained test lipid or bacterial extract. (additional control treatment).

Preparations B, C, D and E provided daily oral doses per mouse of 53 micrograms of total lipids or chloroform/methanol lipid bacterial extract, made up with PBS to a total daily intra-gastric dose per mouse of 0.2 ml.

In the case of preparations C, D and E, lipids or bacterial extract were applied to 1.5 micron diameter approximately spherical particles of silicon dioxide according to the following method:—

Preparation Method for SiO₂ Coating

The suspension matrix was prepared for the silicon dioxide beads via the following steps.

1. Sodium carbonate buffer was prepared

100 ml of 0.05 M NaHCO₃ solution was taken.

The solution pH was adjusted to pH 9.7-9.9 with 0.1 M Na OH solution.

The adjusted buffer was diluted to 200 ml with sterilised water.

2. The suspension matrix was prepared.

50 ml of the Sodium carbonate buffer was taken and to this was added 100 ml of Glycerine

The two were mixed completely

3. The suspension matrix was then autoclaved.

The suspension matrix was then complete.

The lipids or extract were prepared for mixing with the silica via the following steps.

All SiO₂ used in this method was 1.5 micron SiO₂ powder.

4. The extract sample was dissolved and mixed with SiO₂.

The extract sample was weighed into a clean container.

Every 25 mg of extract was dissolved in 1 ml of Chloroform/Methanol (2:1)

The SiO₂ was weighed into the final container. 10 mg of SiO₂ was used for every 5 mg of extract.

The extract solution was added to the SiO₂ and swirled to mix the two.

The solvent was carefully removed by blowing with a stream of dry Nitrogen gas.

The extract was now coating the SiO₂ and was ready for the suspension matrix.

5. The Phospholipid sample was dissolved and mixed with SiO₂.

Every 25 mg of lipid was dissolved in 1 ml of Chloroform/Methanol (2:1)

The SiO₂ was weighed into the final container. 10 mg of SiO₂ was used for every 5 mg of extract.

The lipid solution was added to the SiO₂ and swirled to mix the two.

The solvent was carefully removed by blowing with a stream of dry Nitrogen gas.

The lipid was now coating the SiO₂ and was ready for the suspension matrix.

6. The Glycolipid sample was dissolved and mixed with SiO₂.

Every 25 mg of lipid was dissolved in 1 ml of Chloroform/Methanol (2:1)

The SiO₂ was weighed into the final container. 10 mg of SiO₂ was used for every 5 mg of extract.

The lipid solution was added to the SiO₂ and swirled to mix the two.

The solvent was carefully removed by blowing with a stream of dry Nitrogen gas.

The lipid was now coating the SiO₂ and was ready for the suspension matrix.

7. The Neutral lipid sample was dissolved and mixed with SiO₂

Every 25 mg of lipid was dissolved in 1 ml of Chloroform/Methanol (2:1)

The SiO₂ was weighed into the final container. 10 mg of SiO₂ was used for every 5 mg of extract.

The lipid solution was added to the SiO₂ and swirled to mix the two.

The solvent was carefully removed by blowing with a stream of dry Nitrogen gas.

The lipid was now coating the SiO₂ and was ready for the suspension matrix.

The coated SiO₂ was suspended by the following steps

8. The SiO₂ was suspended by the matrix.

For every 10 mg of SiO₂ (and therefore 5 mg of lipid) 10 ml of the suspending matrix was used.

The suspending matrix was added to the coated SiO₂.

The mixture was shaken to loosen the coated SiO₂ from the walls of the vessel.

The mixture in its sealed container was placed into an ultrasonic bath. The mixture was sonicated for 15 mins. This produced a fine dispersion of the coated SiO₂ within the matrix. The SiO₂ sometimes precipitated to the bottom of the container during this process or over time. Shaking the container re-suspended the SiO₂ into the matrix. If further sonication was required for complete dispersion, sonication was repeated for another 15 mins. It is necessary not to sonicate above the Tₘ (transition temperature) of the lipid. Doing so may result in large multilaminar vesicles rather than coating.

The suspension was now complete and ready for use.

Inmunisation Schedule

There were 6 experimental groups, containing 5 mice each.

200 μl of each preparation (or PBS or Liposome controls) was administered intragastrically to 6 groups of 5 mice, on 5 consecutive days.

On day 7 and day 17, mice from each group were immunised intragastrically with KLH (5 mg) and cholera toxin (10 μg) in 200 μl PBS.

Samples

Saliva samples were collected on day 20, following an intraperitoneal injection of 50 μl pilocarpine (0.5% w/v). On day 21 mice were killed and mesenteric lymph nodes removed.

Preparation of Single Cell-Suspension from Mesenteric Lymph Nodes.

The mice were dissected and mesenteric lymph nodes (MLN) removed and placed in tissue culture medium, RPMI 1640. The MLN from each of the groups were pooled, homogenised and single cell suspension was obtained by passing the solution through a 70 μm nylon mesh strainer.

The cells were then washed three times with RPMI containing 5% foetal calf serum at 4° C., 1200 rpm. The cells were finally suspended in 1 ml of complete media, counted in a haemocytometer with trypan blue to estimate viability and used for proliferation assays.

T-Cell Proliferation Assay

The cells were placed 8×10⁶/ml in triplicate in a 96 well tissue culture plate, with or without KLH at two concentrations 100 μg/ml and 50 μg/ml. The cells were incubated for 5 days at 37° C. and were pulsed with 2 μCi per well of titrated thymidine for the final 15 hours and then harvested onto filters and read in the 96 well matrix scintillation counter. Results were expressed as stimulation index i.e. the activity in antigen or mitogen stimulated wells divided by the activity in control (unstimulated) wells.

ELISA Assay

The specific anti-KLH IgA antibody content of each individual saliva sample was determined in an established ELISA assay and standardised against a positive control antiserum obtained by hyperimmunisation of mice with KLH (100 μg) with cholera toxin (10 μg) in Freund's incomplete adjuvant. Plates were coated with KLH at 2 μg/ml and incubated with four twofold dilutions of samples (100 μl) at 37° C. for 2 hours. The initial dilution of saliva and vaginal samples was 1:10. Sheep anti-mouse IgA was used at 1:1000, for 2 hours at 37° C. Donkey anti-sheep IgG alkaline phosphatase conjugate was used at 1:250 for IgA assays for 1 hour at 37° C. The results were expressed as antibody units calculated from the standard curve obtained from the pooled immune mouse serum given an arbitrary antibody value of 100,000 IgA units/ml. The standard curve was diluted from 1:100 to 1:3200 for IgA samples. The value for each sample dilution falling on standard curve was calculated and the mean taken as the value for the sample.

Results

Cellular Assay

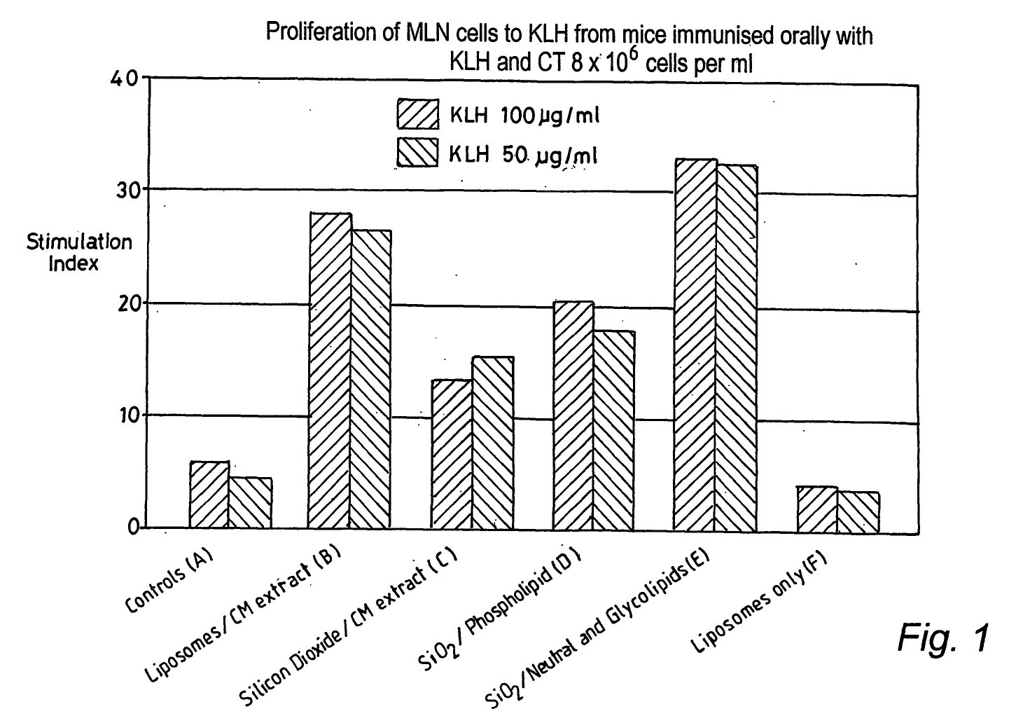

Mesenteric lymph node cells from the 5 mice within the group were pooled. The proliferation of MLN cells to KLH is significant in all the groups compared with controls (FIG. 1). The stimulation index is enhanced in all experimental groups B-E but was particularly prominent for groups B and E. KLH used at the concentration of 100 μh/ml and 50 μg/ml had similar results.

Mucosal Antibodies

Saliva:

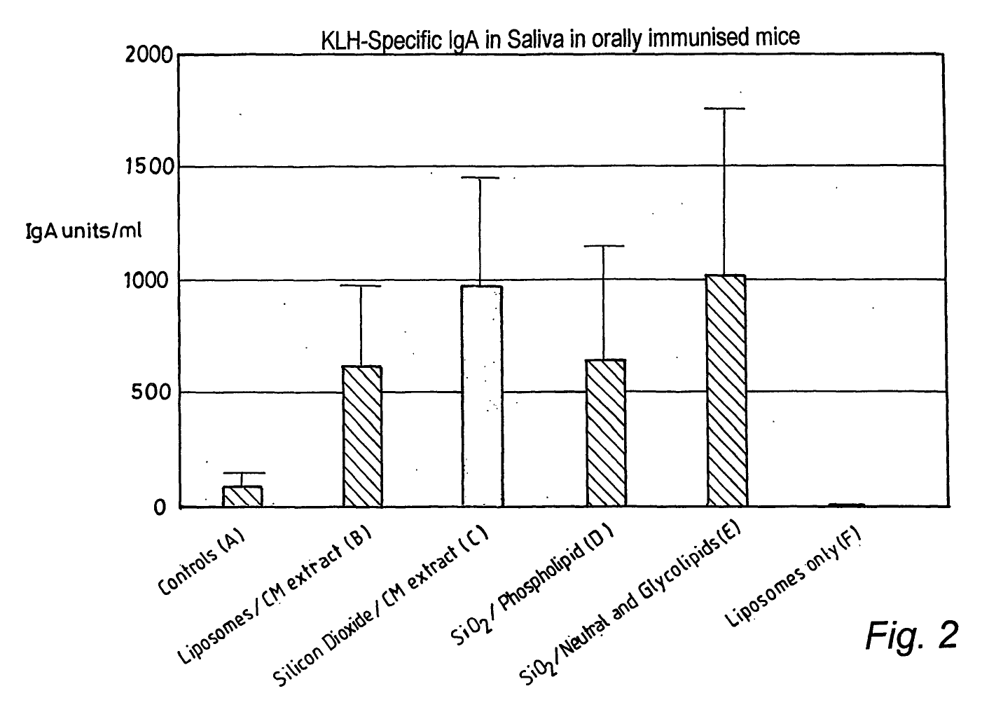

Oral immunisations with antigens resulted in salivary antibodies specific to KLH. Groups B, C, D and E show higher specific salivary IgA antibodies to KLH compared with controls (FIG. 2).

Conclusions from Example 4

1. Results confirm that the lipid bacterial extracts, given intra-gastrically in liposomes from 7 to 2 days before first immunisation, enhanced the cellular and antibody immune response.

2. The method of presenting the lipids (both extract and synthesised lipid) by coating them onto particles of silicon dioxide, before intra-gastric administration, has been shown to be effective in enhancing immune response to antigens.

3. Synthetic phospholipids (11 given simultaneously) and synthetic neutral and glycolipids (8 given simultaneously) have been shown to be effective in enhancing immune response to antigens, when given by the methods described.

### EXAMPLE 5

This is a comparison of treatments in Example 2 (Test A) and Example 3 (Test B).

Comparison is made between two tests in mice where MLN T-cell proliferation response to KLH was assayed by the method described in Example 4 (Cellular assay).

In both tests, Bacillus subtilis was grown in a clear liquid medium containing yeast extract, sucrose, magnesium and other soluble nutrients, according to well established procedures. The cells were separated from the culture by centrifugation, subjected to partial autolysis and lipid was extracted by agitation for 48 hours in a mixture of 2;1 chloroform and methanol. Following centrifugation, the supernatant was concentrated by evaporation to yield lipid extract.

In Test A the lipid extract was dispersed in an aqueous medium using a commercially available emulsifying agent (Crillet 4) and administered to 5 mice by gastric lavage, daily for 5 consecutive days, at a dose rate per mouse per day of 53 micrograms of extract on 0.2 ml of PBS.

In Test B, lipid extract was prepared and administered to mice of the same age and strain (young female Balb C) by the same route and at the same dosage, but incorporated in liposomes of the bilosome type (method of J. Brewer, Strathclyde University) of approximately 1-3 microns diameter.

In both tests similar groups of control mice received daily intra-gastric administrations of 0.2 ml PBS.

On days 7 and 25 mice from both treated and control groups in both tests were immunised intragastrically with KLH (5 micrograms) and cholera toxin (10 micrograms) as in Example 1.

Mesenteric lymph nodes were removed on day 21, and tests of T-cell proliferative response to KLH were performed as in Example 1.

Results were determined in terms of 3H thymidine incorporation, and expressed in CPM above backgrounds.

In Test A, where the lipid extract was given in non-particulate form, cells from the treated mice showed no proliferative response after 3 days incubation, compared with 37,000 cpm above background for controls. In this case, the T-cell proliferative response was therefore strongly depressed by the treatment.

In Test b, where the lipid extract was given in particulate form in liposomes, the treated mice showed a proliferative response after 3 days incubation of 140,000 cpm above background, compared with 12,000 cpm for controls. In this case, the T-cell proliferative response was clearly strongly increased by the treatment.

This example shows that the effect of lipids applied to mucosal surfaces upon the animal's immune response to antigens is profoundly affected by the physical from in which the lipids are administered. Depending upon the physical from, the immune response to antigens may be increased or suppressed.

### EXAMPLE 6

In a further test, two groups of mice as employed in Example 2, were immunised by two administrations of antigen, 14 days apart, by the intraperitoneal and intragastric routes.

These administrations were accompanied by concurrent administrations of Group 2 lipids (glycolipids and neutral lipids) as employed in Example 4.

Materials employed use:

| | |
|---|---|
| Intraperitoneal administration | |
| Synthetic lipids as in Example 4, | 0.14 microgram in 50 mcl aqueous |
| Group 2 | dispersion |
| KLH | 0.5 micrograms in 50 mcl aqueous |
| | dispersion |
| Intragastric administration | |
| Synthetic lipids as in Example 4, | 14 micrograms on SiO₂ particles |
| Group 2 | in 0.2 ml PBS |
| KLH | 5 milligrams in 0.2 ml PBS |

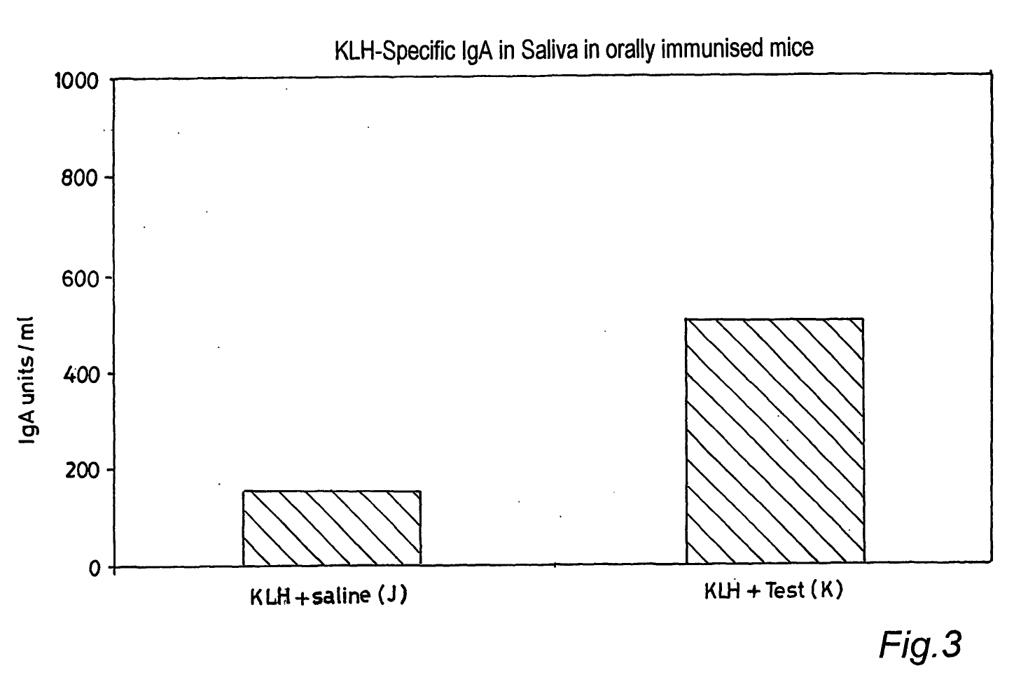

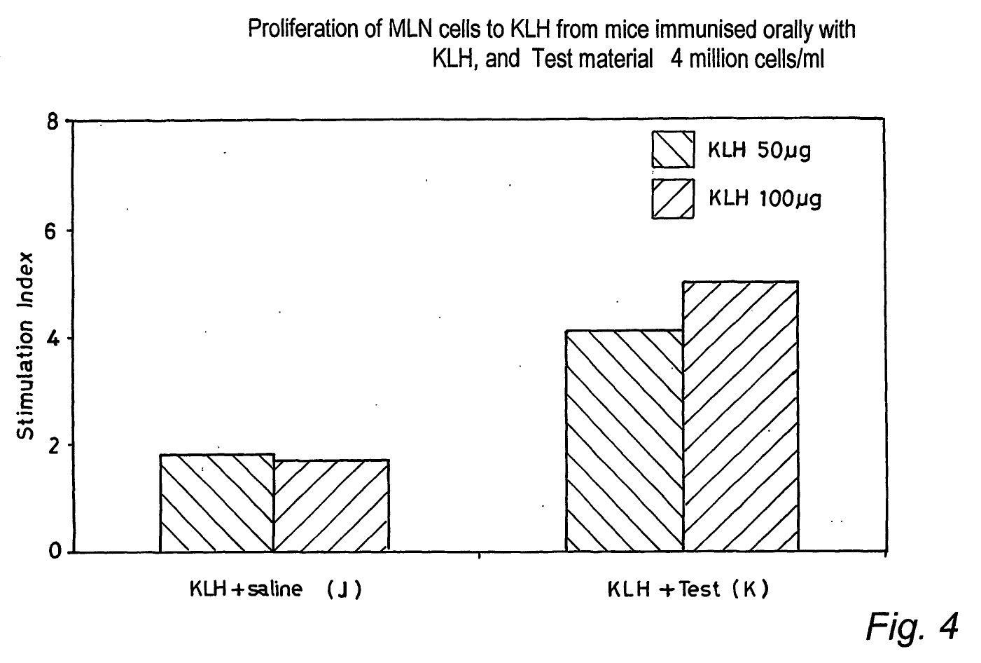

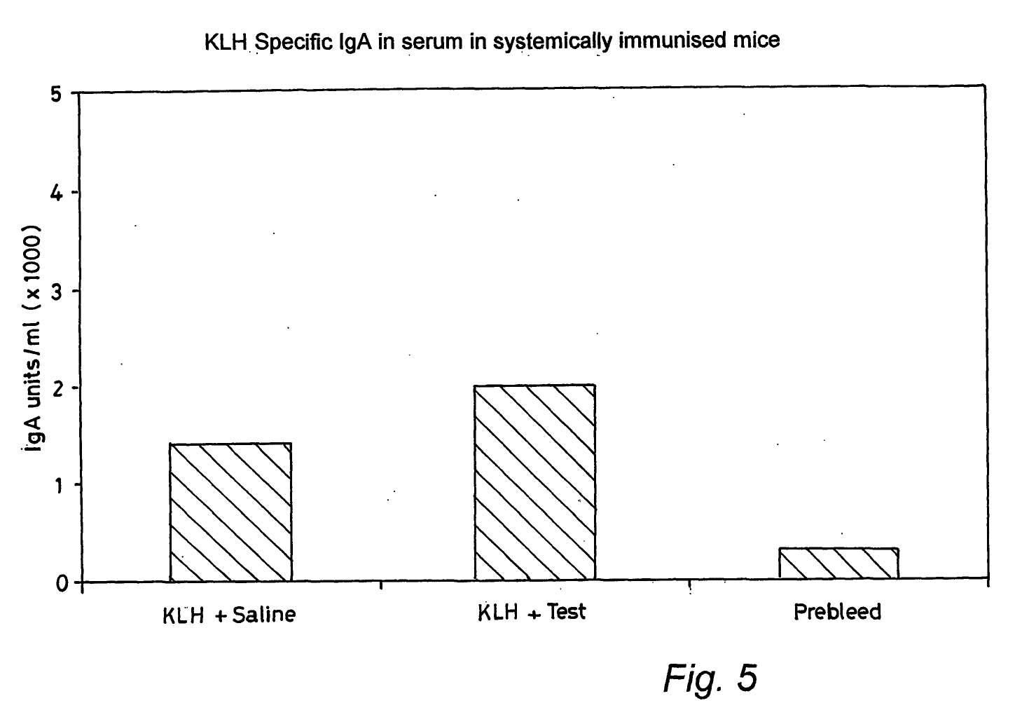

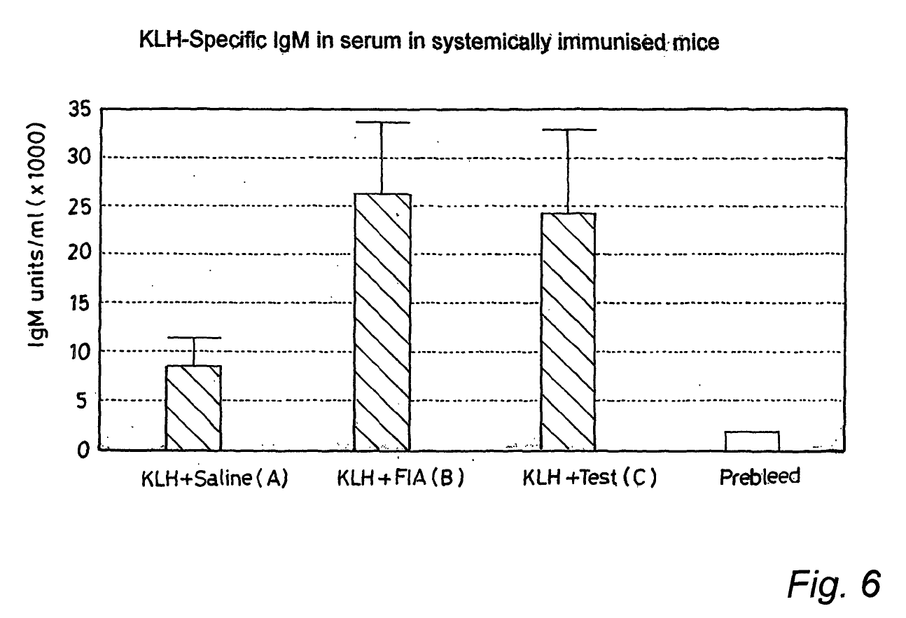

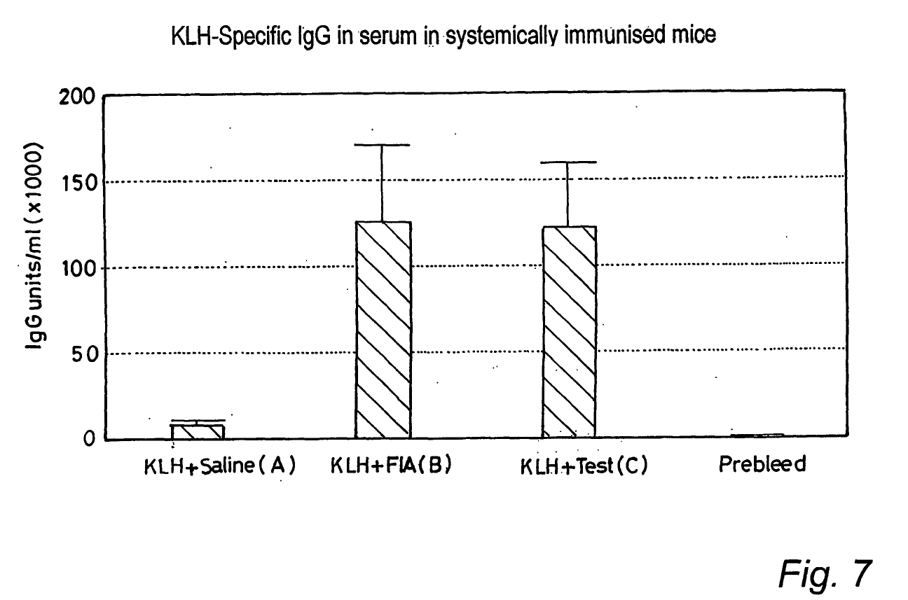

Results of Example 6 are given in FIGS. 3, 4 and 5

The results show that Group 2 lipids can be employed as adjuvants, given concurrently with antigens, both parenterally and via the gastro-enteric mucosa.

### EXAMPLE 7

The objective of this study was to investigate the adjuvanticity of 19 different lipids.

Materials and Methods

Mice

Female Balb/c mice, 6-8 weeks and weighing about 20-22 gms were used and maintained on a normal mouse diet throughout the study.

Lipid Preparations

The following lipid preparations were prepared:—

List of abbreviations:

| | | |
|---|---|---|
| C | PAM | (L-α-Phosphatidicacid, dimyristoyl) |
| D | PAH | (L-α-Phosphatidicacid, diheptadecanoyl) |
| E | PCM | (DL-α-Phosphatidylcholine, dimyristoyl) |
| F | PCP | (L-α-Phosphatidylcholine, dipentadecanoyl) |
| G | PCH | (L-α-Phosphatidylcholine, diheptadecanoyl) |
| H | PCS | (DL-α-Phosphatidylcholine, distearoyl) |
| I | PEM | (L-α-Phosphatidylethanolamine, dimyristoyl) |
| J | PEP | (DL-α-Phosphatidylethanolamine, dipalmitoyl) |
| K | PES | (L-α-Phosphatidylethanolamine, distearoyl) |
| L | PEH | (L-α-Phosphatidylethanolamine, diheptadecanoyl) |
| M | PSP | (DL-α-Phosphatidyl-L-serine, dipalmitoyl) |
| N | PS | (N-Palmitoyl-d-sphigosine) |
| O | PC | (N-Palmitoyl dihydroglucocerebroside) |
| P | TM | (Trymyristin) |
| Q | Tpen | (Tripentadecanoin) |
| R | Tpal | (Tripalmitin) |
| S | DPM | (1,2 Diplamitoyl-3-myristoyl-rac-glycerol) |
| T | DSM | (1,2 Distearloyl-3-myristoyl-rac-glycerol) |
| U | DSP | (1,2 Distearoyl-3-palmitoyl-rac-glycerol) |
| V | MIX | (Cocktail of equal amounts of each of the above lipids) |

Experimental Groups and Immunisation Schedule

There were 22 groups of mice marked alphabetically from A-V. Group A (8 mice) was the negative control (KLH+Saline), Group B (8 mice) was the positive control (KLH+IFA). Groups C-V were the experimental groups containing 6 mice each. 100 μl of antigen preparation comprising of 5 μl lipid+45 μl saline+50 μl (2 mg/ml KLH) was administered to 20 groups of 6 mice each, on week 0 followed by a boost on week 2.

Samples

On day 7 and day 21 after the booster dose, the mice were tail bled, the serum separated, aliquoted and stored at 4° C.

ELISA Assay

The specific anti-KLH IgG and IgM antibody content of each individual serum sample was determined in an established ELISA assay and standardised against a positive control antiserum obtained by hyperimmunisation of mice with KLH (100 μg) in Freund's incomplete adjuvant. Plates were coated with KLH at 2 μg/ml and incubated with four twofold dilutions of samples (100 μl) at 37° C. for 2 hours. The initial dilution of serum samples was 1:50 for IgA assay and 1:100 for IgG and IgM assays. Sheep and mouse IgA and IgM were used at 1:1000 and IgG at 1:2000 for 2 hours at 37° C. The results were expressed as antibody units calculated from the standard curve obtained from pooled immune mouse serum given an arbitrary antibody value of 100,000 IgA units/ml and 1×10⁶ IgG and IgM units/ml. The standard curve was diluted from 1:100 to 1:3200 for IgA, 1:4000 to 1:256,000 for IgG and 1:1000 to 1:32000 for IgM samples. The value for each sample dilution falling on standard curve was calculated and the mean taken as the value for the sample.

Results

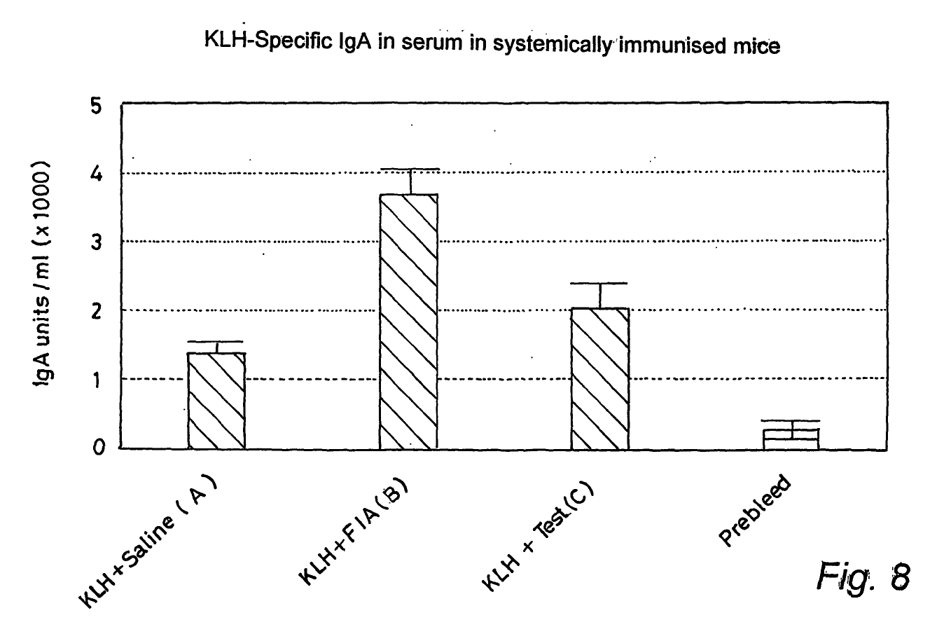

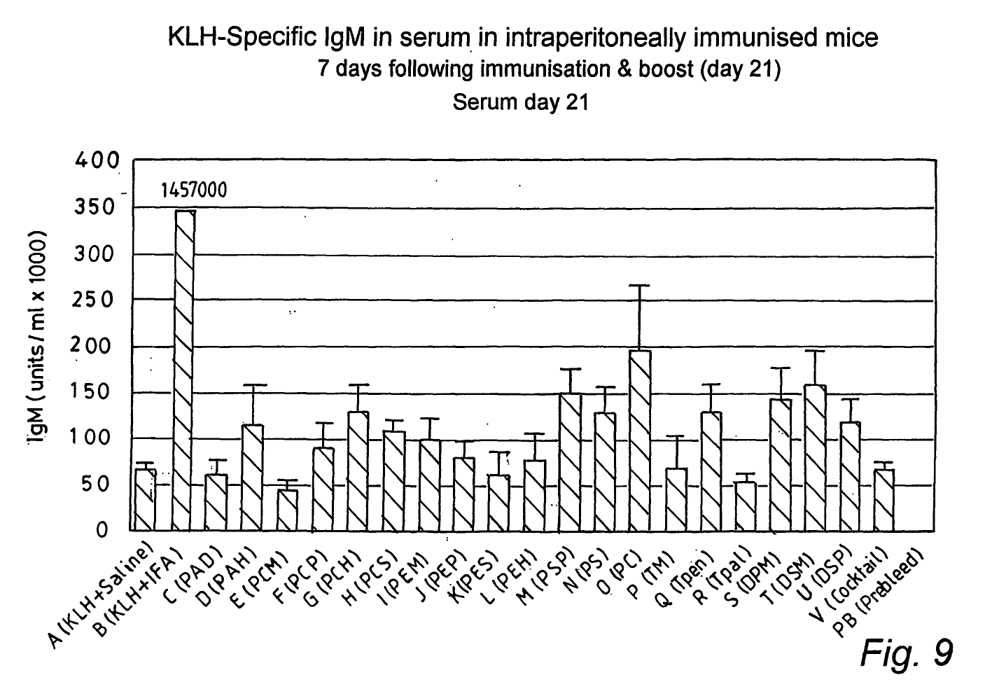

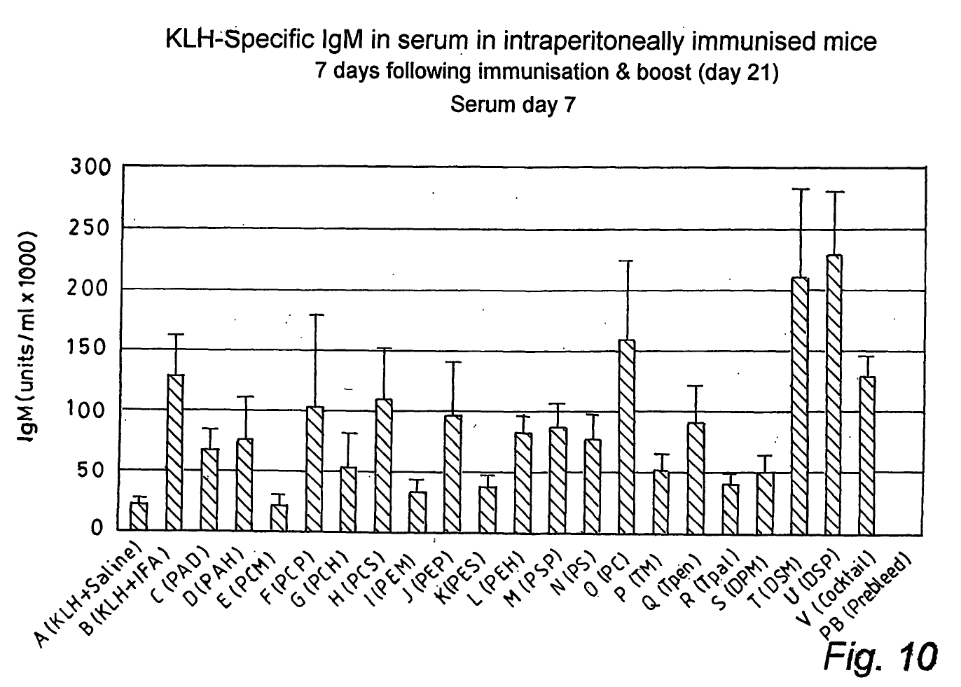

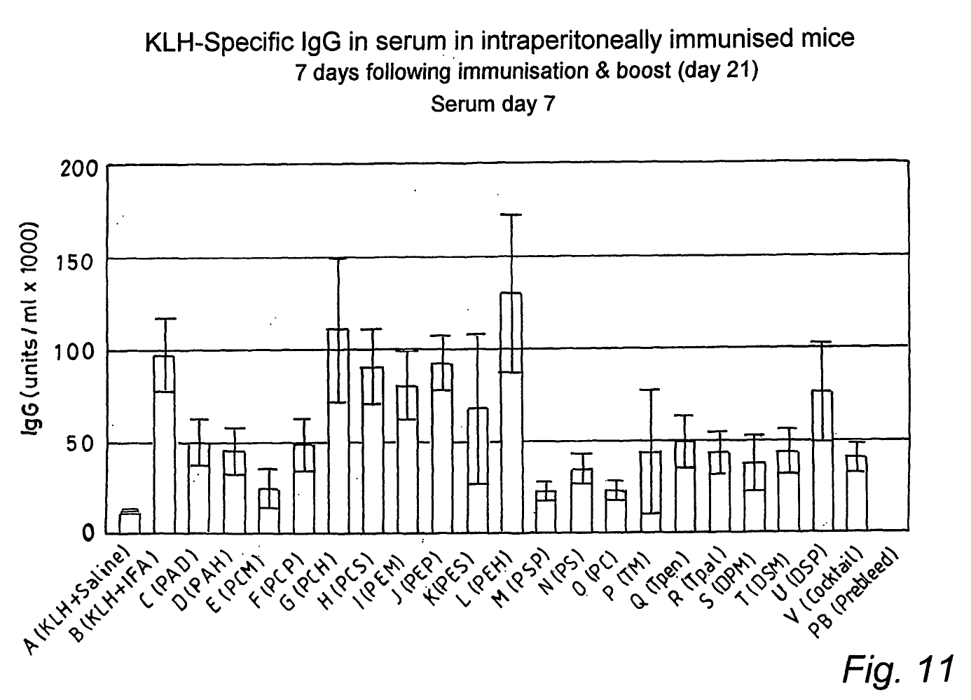

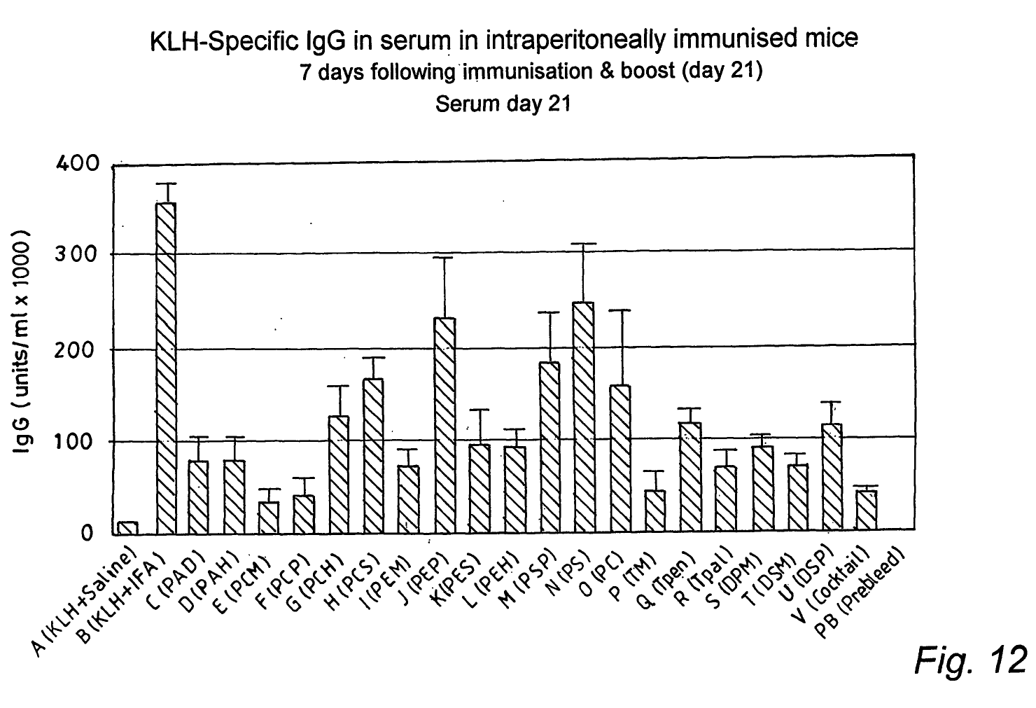

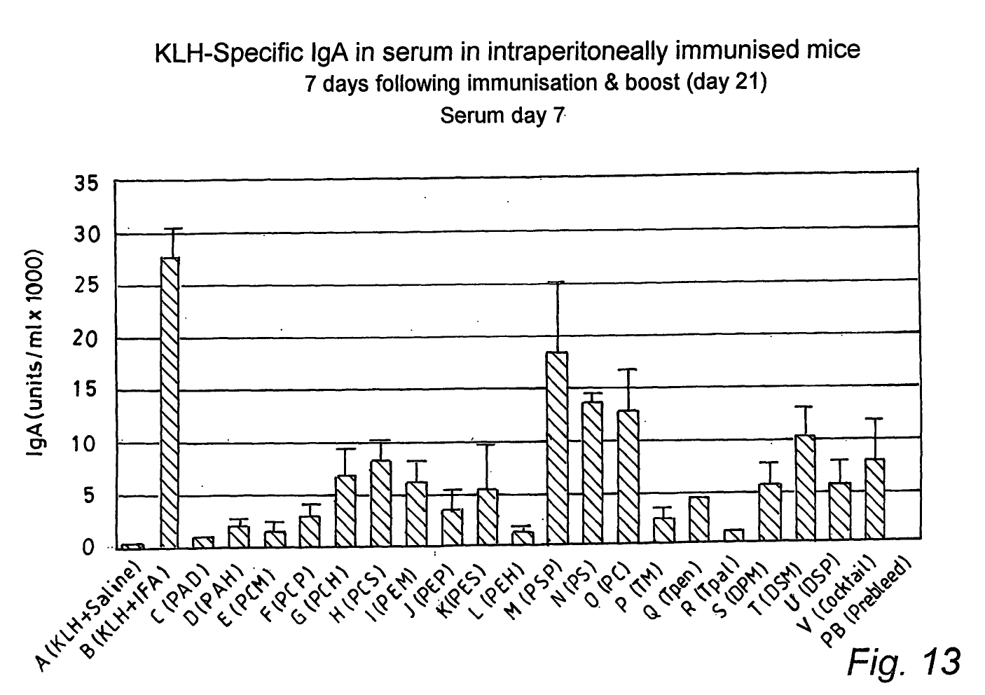

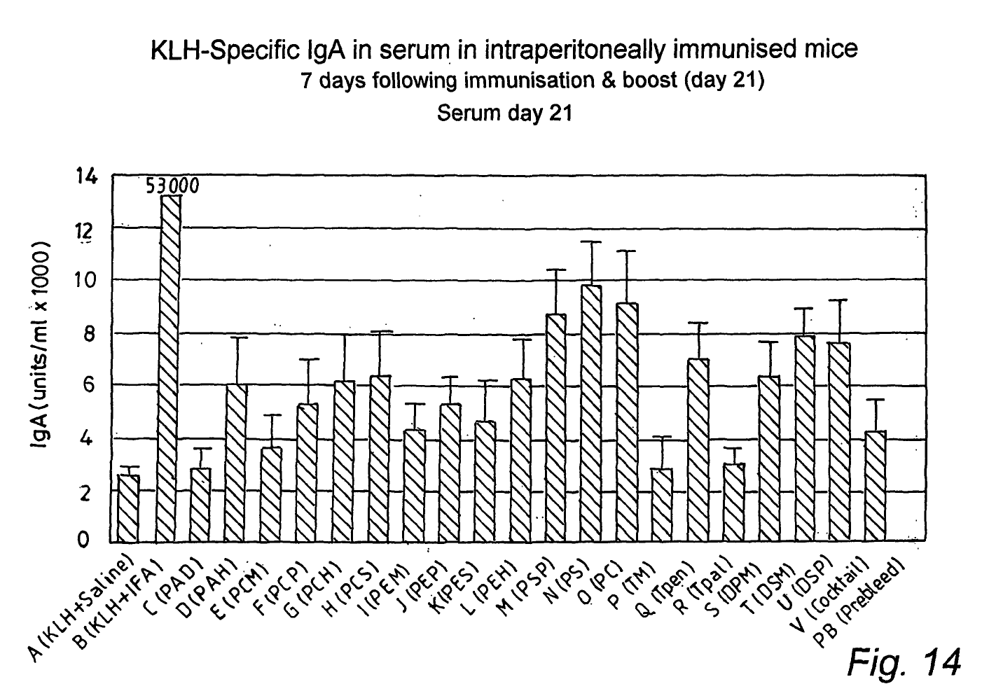

Results are given in the form of histograms (FIGS. 9 to 14), plotting the Ig response (units/ml) against the trial grouping. From these data each trial group was given a numerical score based upon its performance compared to the control. The number signifies multiples of the control response.

| | Day 7 Results | | | Day 21 Results | | | Ranking |
|---|---|---|---|---|---|---|---|
| | IgA | IgG | IgM | IgA | IgG | IgM | Total |
| KLH + Saline | 1 | 1 | 1 | 1 | 1 | 1 | 6 |
| KLH + FIA | 72 | 9 | 3 | 20 | 26 | 23 | 153 |
| PAM | 4 | 5 | 1 | 1 | 6 | 1 | 18 |
| PAH | 5 | 4 | 2 | 2 | 6 | 2 | 21 |
| PCM | 4 | 2 | 1 | 1 | 3 | 1 | 12 |
| PCH | 18 | 10 | 1 | 2 | 10 | 2 | 43 |
| PCP | 9 | 5 | 2 | 2 | 3 | 1 | 22 |
| PCS | 22 | 8 | 2 | 2 | 13 | 2 | 49 |
| PEM | 17 | 7 | 1 | 2 | 6 | 2 | 35 |
| PEH | 4 | 12 | 2 | 2 | 7 | 1 | 28 |
| PEP | 9 | 8 | 2 | 2 | 17 | 1 | 39 |
| PES | 15 | 6 | 1 | 2 | 7 | 1 | 32 |
| PSP | 47 | 2 | 2 | 3 | 14 | 2 | 70 |
| PS | 35 | 3 | 2 | 4 | 19 | 1 | 64 |
| PC | 33 | 2 | 3 | 3 | 12 | 3 | 56 |
| TM | 6 | 4 | 1 | 1 | 3 | 1 | 16 |
| TPeN | 11 | 4 | 2 | 3 | 9 | 2 | 31 |
| TPaL | 3 | 4 | 1 | 1 | 6 | 1 | 16 |
| DPM | 16 | 3 | 1 | 2 | 7 | 2 | 31 |
| DSM | 26 | 4 | 4 | 3 | 5 | 3 | 45 |
| DSP | 15 | 7 | 5 | 3 | 8 | 2 | 40 |
| MIX | 21 | 3 | 3 | 2 | 3 | 1 | 33 |

Results

| | | | | | Head gr up | | Head |
|---|---|---|---|---|---|---|---|
| | | | | Total | Mass | Head | group |
| Ranked | | | Head | Partial | (AMU) | group | Charge:Vol |
| High to | Chain | Optical | group | Charge | Atomic Mass | Volume | (eV/ |
| Low | Lengths | isomers | Charges | (eV) | Units | (cm⁻³mol⁻¹) | cm⁻³mol⁻¹) |
| KLH + FIA | | | | | | | |
| PSP | 18/18/18 | dl | −ve; −ve; −ve | −1.044 | 313.165 | 195.6 | −0.005 |
| PS | 20/20/20 | d | −ve | −0.305 | 119.123 | 84.6 | −0.004 |
| PC | 20/20/20 | | −ve; −ve; +ve | −1.148 | 281.269 | 199.2 | −0.006 |
| PCS | 20/20/20 | dl | −ve; −ve; +ve | −1.876 | 325.262 | 216.3 | −0.009 |
| DSM | 20/20/14 | | −ve | −0.492 | 218.211 | 155.4 | −0.003 |
| PCH | 17/17/17 | l | −ve; −ve; +ve | −1.876 | 325.262 | 216.3 | −0.009 |
| PEP | 18/18/18 | dl | −ve; −ve; +ve | −1.015 | 269.154 | 167.5 | −0.006 |
| DSP | 20/20/18 | | −ve | −0.492 | 218.211 | 155.4 | −0.003 |
| PEM | 14/14/14 | l | −ve; −ve; +ve | −1.015 | 269.154 | 167.5 | −0.006 |
| MIX | | | | | | | |
| PES | 20/20/20 | l | −ve; −ve; +ve | −1.015 | 269.154 | 167.5 | −0.006 |
| TPeN | 15/15/15 | | −ve | −0.492 | 218.211 | 155.4 | −0.003 |
| DPM | 18/18/14 | | −ve | −0.492 | 218.211 | 155.4 | −0.003 |
| PEH | 17/17/17 | l | −ve; −ve; +ve | −1.015 | 269.154 | 167.5 | −0.006 |
| PCP | 18/18/18 | | −ve; −ve; +ve | −1.876 | 325.262 | 216.3 | −0.009 |
| PAH | 17/17/17 | l | −ve | −0.28 | 240.112 | 147.1 | −0.002 |
| PAM | 14/14/14 | l | −ve | −0.28 | 240.112 | 147.1 | −0.002 |
| TM | 14/14/14 | | −ve | −0.492 | 218.211 | 155.4 | −0.003 |
| TPaL | 18/18/18 | | −ve | −0.492 | 218.211 | 155.4 | −0.003 |
| PCM | 14/14/14 | dl | −ve; −ve; +ve | −1.876 | 325.262 | 216.3 | −0.009 |
| Control | | | | | | | |

Observations

The trends within the test are as follows:

Chain Uniformity

When multiple chains are present on a lipid it is preferable to have at least two different lengths on the same lipid molecule.

This is shown by comparison of DSP, DSM against TpaL and TM. Both DSP and DSM have mixed chain lengths, and the resulting performance is good (Ranking of 39 and 45 respectively). TpaL and TM each have three fatty acid chains that are the same length. These are comparable in length to those on DSP and DSM. Their performance in the trial was poor (Ranking of 15 and 17 respectively).

Optical Isomerism

When a lipid shows optical isomerism it is preferable to use the d isomer.

This can be shown by the comparison of PEP against PEH. Both are structurally similar, however PEP, which contains a d isomer, performs better than PEH that only contains an l isomer (Ranking 40 and 28 respectively).

Chain Length

The length of the fatty acid chain is important as a governing principle. It is preferable to have a chain length that consists of more than 14 carbon atoms and ideally 18 to 20 carbon atoms.

This can be shown by the comparison of PEP and PSP against PAM and PCM. Both PEP and PSP have fatty acid chains containing 18 carbon atoms and both perform well (Ranking 40 and 70 respectively); PAM and PCM both have chains that contain 14 carbon atoms and perform poorly in the experiment (Ranking 18 and 12 respectively).

Head Group Structure

The size of the head group is important. It is preferable to have a large (both in volume and mass) head group. Ideally the mass should be 269 AMU or above and the volume should be 167 cm⁻³mol⁻¹ or above.

This can be shown by the comparison of PEP and PSP against PAM and TM. Both PEP and PSP have large head groups. Both these lipids perform well (Ranking 40 and 70 respectively). Both PAM and TM have smaller head groups; both of these lipids perform poorly (Ranking 18 and 17 respectively).

The charge of the head group is important. It is preferable for the head group to have a combined partial charge of −1.015 eV or more strongly negative. Ideally the combined partial charge should be −1.044 eV or more strongly negative.

This can be shown by the comparison of PEP and PSP against PAM and TM. Both PEP and PSP have strongly negative head groups; both these lipids perform well (Ranking 40 and 70 respectively). Both PAM and TM have weakly charged head groups; both of these lipids perform poorly (Ranking 18 and 17 respectively).

Where the head group has a partial charge it is preferable for the charge to be in multiple centres.

This can be shown by the comparison of PEP and PSP against PAM and TM. Both PEP and PSP have head groups with multiple charge centres. Both these lipids perform well (Ranking 40 and 70 respectively). Both PAM and TM have head groups with a single charge centre; both of these lipids perform poorly (Ranking 18 and 17 respectively).

The head group charge to volume ratio should be relatively large. It is preferable to have a charge of −0.004 eV/cm⁻³mol⁻¹ or more per unit. Ideally the charge to volume ration should be −0.006 eV/cm⁻³mol⁻¹ or more strongly negative.

This can be shown by the comparison of PEP and PSP against PAM and TM. Both PEP and PSP have head groups with stronger negative charge to volume ratios. Both these lipids perform well (Ranking 40 and 70 respectively). Both PAM and TM have head groups with weak negative charge to volume ratios; both of these lipids perform poorly (Ranking 18 and 17 respectively).

### EXAMPLE 8

The objects of this experiment were:

a) To test the effects of unsaturated lipids compared to closely similar saturated analogues on enhancing immune response to antigens.

b) To test the effect of naturally occurring lipids of plant origin, administered orally in particulate form, on enhancing said response.

Materials and Methods

7 groups of 6 mice as employed in Example 4, were immunised by three administrations of test lipids on three consecutive days (Days 1, 2 and 3). On Dyas 7 and 14 mice were immunised intragastrically with KLH (5 mg) in 200 μl saline containing cholera toxin (10) as employed in Example 4.

Materials Employed:—

Intragrastric Lipid Administration

Synthetic lipids as in 4 (See Fig, PHOS.) 40 μg on SiO₂ particles in 0.2 ml PBS, per mouse per day

Phosphatidylcholine, distearoyl (PCS) 40 μg on SiO₂ particles in 0.2 ml PBS, per mouse per day

Phosphatidylcholine, dioleoyl (PCO) 40 μg on SiO₂ particles in 0.2 ml PBS, per mouse per day

Trimyristin (TM) 40 μg on SiO₂ in 0.2 ml PBS, per mouse per day

Trilinolenin (TL) 40 μg on SiO₂ particles in 0.2 ml PBS, per mouse per day

Coconut, Palm and Cottonseed Oil (COM.) 40 μg on SiO₂ particles in 0.2 ml PBS, per mouse per day

The mice were killed on Day 21. Samples of mesenteric lymph nodes were taken for assays of proliferative cellular response to KLH.

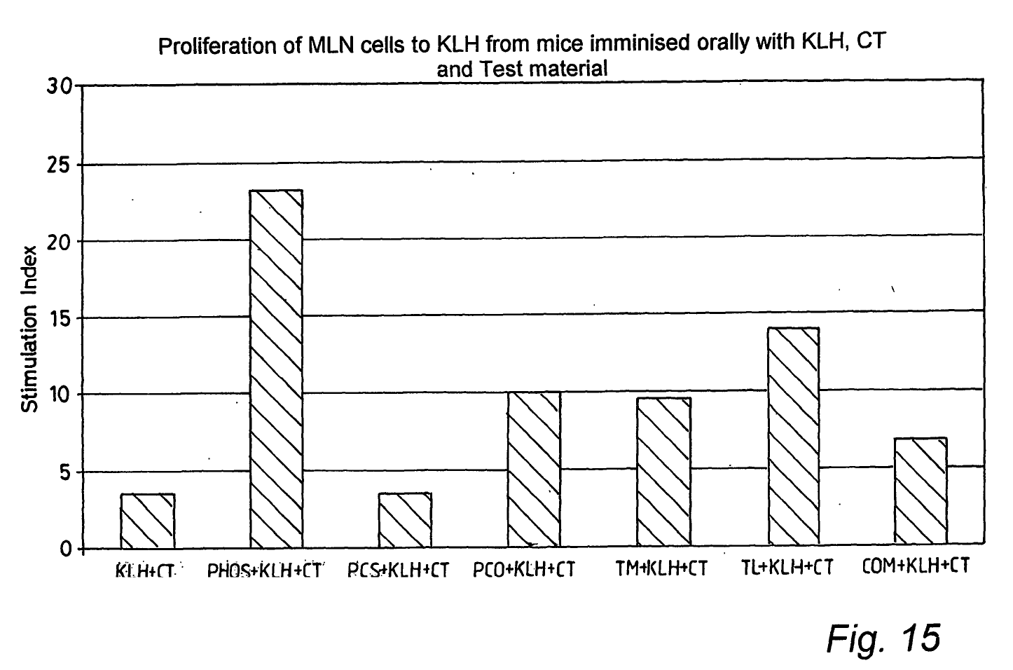

Results are given in FIG. 15.

Comparison of the control treatment receiving no lipid pre-treatment KLH+CT with the positive control receiving phospholipid pre-treatment PHOS+KHL+CT) previously shown to be effective in Example 4) confirms validity of this test. The results show camparison between Phosphatidycholine, distearloyl, a saturated lipid that showed good performance in Example 7, and Phosphatidycholine, dioleoyl, as structurally similar unsaturated analogue. Also compared within these results are Trimyristin, a saturated lipid that showed poor performance in Example 7, and Trilinolenin, a structurally similar unsaturated analogue. Both of these comparisons show that the unsaturated analogues performed better than their unsaturated equivalents giving a strong proliferative response compared to control. These results therefore confirm the effectiveness of unsaturated lipids in enhancing the immune response. Also, this example shows that lipids of plant origin are useful in achieving this purpose.

Trial group COM, receiving plant based lipids selected to give comparable content to synthetic lipids as employed in Example 4, Group 1, showed a strong proliferative response when compared to the control group. In this case the T-cell proliferative response was strongly increased by the treatment.

1. Use of a lipid, as hereinbefore defined, having a glycerol backbone carrying at least one alkyl or acyl chain, wherein the lipid is phospholipid, glycolipid or neutral lipid, saturated or unsaturated, and the number of carbon atoms in the hydrocarbon tails lies between 10 and 22 inclusive, in the preparation of a composition to stimulate and/or modulate an immune response in a human or animal subject.

2. A use as claimed in claim 1, wherein the composition comprises any one or a mixture of one or more of the aforesaid phospholipid, glycolipid or neutral lipid which may be synthesised, produced by microbial fermentation, or may be contained in or derived from natural materials such as animal or vegetable oils from which they may or may not be extracted or concentrated as by distillation, fractionation, phoresis or other known processes.

3. A use as claimed in claim 1 or claim 2, wherein the composition is adapted to be given to man or animals in non-particulate or particulate form, by parenteral or transmucosal administration, for the purpose of modulating an immune response to an antigen, wherein the lipid/s present in the composition is characterised by any one or more of the following:—
a) one or more fatty acid chains, preferably two or more;
b) a chain length of 10 to 22 carbon atoms, preferably 12 to 20 and more preferably 16 to 20 or even 18 to 20;
c) where there are two or more chains, the chains are of equal length or, preferably, of unequal length;
d) where a lipid shows optical isomerism, the d or l isomer is employed, but preferably the d isomer;
e) where a lipid has a head group, the head has a mass of at least 269 Atomic Mass Units (AMU) and a volume of at least 167 cm⁻³mol⁻¹;
f) where a lipid has a head group, it has a combined partial electrical charge of −1.015 eV or more strongly negative, and preferably of −1.044 eV or more strongly negative;
g) where a head group has a charge, it may have a single centre, but preferably has multiple centres;
h) a head group charge to volume ratio is preferably −0.004 eV/cm⁻³mol⁻¹ or more strongly negative, and more preferably −0.006 eV/cm⁻³mol⁻¹ or more strongly negative; and
i) the lipids are saturated or unsaturated.

4. A use as claimed in claim 1 or 2, wherein the composition is adapted to be administered trans-mucosally, or parenterally.

5. A use according to claim 3 or 4, wherein the composition is adapted to be administered to be administered trans-mucosally and comprises lipid in particulate form.

6. A use according to claim 5, wherein the composition comprises lipid/s derived from bacterial cells which have been subjected to a degradation step to render the lipid component thereof available to receptive areas of the gut wall or other mucosal surface, the degradation step being controlled so that the size of the resulting lipid-bearing particulate components, separately or in association with other materials, lies within predetermined limits.

7. A use according to claim 6, wherein the degradation step is effected by use of an enzyme, by autolysis, by heat treatment, such as boiling, autoclaving, irradiation (e.g. gamma irradiation) or microwaving, or by chemical treatment.

8. A use according to claim 6 or claim 7, wherein the particle size is determined by known methods, such as microscopic examination or by sizing techniques employing beams of radiation such as laser beams, or by sizing instruments such as the Malvern Mastersizer.

9. A use according to any one of claims 6, 7 or 8, wherein the particle size of lipid-containing degraded or partially degraded bacteria is at least partially controlled by known centrifugation techniques and further controlled by standard filtration procedures, so that particles of sizes outside the predetermined limits are largely or completely excluded.

10. A use according to claim 9, wherein particles of differing weights or densities are separated by centrifugation.

11. A use according to any one of claims 6 to 10, wherein the predetermined particle size limits on lipids containing degraded or partially degraded bacteria are 0.1 microns to 100 microns, more specifically 0.2 to 25 microns, preferably 0.2 microns up to 15 microns or even 0.3 up to only 10 microns and ideally 0.4 to 8 microns or 0.5 to 5 microns.

12. A use according to any one of claims 1 to 5, wherein the composition comprises lipid consisting of natural materials, or obtained therefrom by means such as extraction with solvents such as chloroform and methanol, or alternatively the lipid is of synthetic origin; the lipid being rendered into a particulate form by incorporation into or onto liposomes or bilosomes or other appropriately sized particulate matter, such as silica, titanium dioxide, or kaolin particles, or other such material capable of absorbing or adsorbing lipids.

13. A use according to claim 12, wherein the lipid or lipids are rendered into a liquid of low viscosity, as by heating, dissolving in solvents such as chloroform or methanol or by forming into an aqueous preparation by means of an emulsifying agent, to form a emulsion or colloid, and particles to be coated are then dispersed in the liquid by stirring, shaking vibration, sonication or other known means of dispersion, the liquid then being removed to leave a coating of the lipid or lipids on the particle surfaces.

14. A use according to claim 13, wherein the liquid is removed by evaporation of solvents or aqueous medium, centrifugation (batch or continuous flow) or filtration, preferably cross-flow filtration.

15. A use as claimed in claim 12 or claim 13, wherein the lipid is shown, for instance by microscopic examination, to be applied to the particles, and a carrier may be added in suitable volume to provide a convenient dosage form.

16. A use according to any one of claims 12 to 14, wherein the lipid bacterial extracts or lipid-containing degraded bacteria or non-bacterial lipids, whether or not incorporated into liposomes, bilosomes or attached to suitably sized particles, are incorporated into particulate formulations of wet or dry powder or granular mixes, or alternatively incorporated into liquids such as water, oil, milk or any beverage.

17. A use according to any one of claims 12 to 14, wherein powder formulations are administered as tablets, capsules or paste, or incorporated into foods.

18. A use according to any one of claims 1 to 11, wherein the lipid bacterial extracts or lipid-containing partially degraded bacteria are mixed with oil, which may be dispersed in water or other suitable liquid with or without the aid of emulsifying agents such as Crillet 4, thereby forming an emulsion in which the bacterial extracts or degraded bacterial product is contained within or bound to the dispersed phase.

19. A use according to claim 14, wherein the carrier particles have a size range of 0.1 to 100 microns, preferably 0.1 to 25 microns and most preferably 0.2 to 15 or even 0.3 to 10 microns, and ideally 0.4 to 8 microns or 0.5 to 5 microns.

20. A use according to claim 3, wherein the composition may be administered parenterally or trans-mucosally in particulate form, with particles of dimensions within the limits of claim 11.

21. A use according to any one of claims 1 to 20, wherein the lipids are phospholipids, glycolipids or neutral lipids, saturated or unsaturated, with or without branching in their fatty acid chains, within the categories of:—
Phosphatidic acids
Phosphatidylethanolamines
Phosphatidylserines
Phosphatidylinositols
Phosphatidylcholines
Cardiolipins
Triglycerides
1,2-Diglycerides
1,3-Diglycerides
Monoglycerides
and their isomers

22. A use according to claim 21, wherein the lipids include any one or more of the following:—
Phosphatidylcholine, dioleoyl
Trilinolenin
L-α-Phosphatidicacid,
Dimyristoyl L-α-Phosphatidicacid,
Diheptadecanoyl DL-α-Phosphatidylcholine,
Dimyristoyl L-α-Phosphatidylcholine,
Dipentadecanoyl L-α-Phosphatidylcholine,
Diheptadecanoyl DL-α-Phosphatidylcholine,
Distearoyl L-α-Phosphatidylethanolamine,
Dimyristoyl DL-α-Phosphatidylethanolamine,
Dipalmitoyl 1,2-Distearoyl-sn-glycero-3-phosphoethanolamine
L-α-Phosphatidylethanolamine,
Diheptadecanoyl DL-α-Phosphatidyl-L-serine,
Dipalmitoyl N-Palmitoyl-d-sphingosine
N-Palmitoyl digydroglucocerebroside
Trymyristin
Tripentadecanoin
Tripalmitin
1,2 Dipalmitoyl-3-myristoyl-rac-glycerol
1,2 Distearoyl-3-myristoyl-rac-glycerol
1,2 Distearoyl-3-palmitoyl-rac-glycerol
rac-1,2-Dimyristoyl-3-palmitoylglycerol

23. A method of promoting and/or modifying an immune response in a human or animal subject, the method comprising administering to the subject a composition prepared by the method of any one claims 1-22.

24. A method according to claim 23, wherein the lipid or lipids are administered transmucosally in particulate form, or given parenterally in either particulate or non-particulate form.

25. A method according to claim 24, wherein trans-mucosal administration is achieved by the oral route, by buccal or sub-lingual placement or spray, or by intranasal spray.

26. A method according to any one of claims 23, 24 or 25, wherein the lipid or lipids are administered together with an antigen or antigens, as an adjuvant, or given separately, especially prior to the antigenic challenge, in order enhance the immune response.

27. A method according to any one of claims 23, 24 or 25, for enhancing the immune response to an antigen, wherein the lipid or lipids are administered together with the antigen(s) to which an enhanced immune response is desired.

28. A method according to any one of claims 23, 24 or 25 for enhancing the immune response to an antigen, wherein the lipid or lipids are administered separately, preferably in advance, of the antigen(s) to which an enhanced immune response is desired.

29. A method according to any one of claims 23 to 25, wherein for the purpose of immune modulation, such as where the intention is to suppress rather than enhance the immune response to antigenic stimulus, for instance, in the control of immune diseases, the lipid materials are given trans-mucosally, but in non-particulate form.

30. A method according to claim 29, wherein the lipid/s is administered dispersed in liquid emulsion, colloid, suspension, solution or other liquid dispersion or in a gel, paste or is dispersed in solid form incorporating e.g. starch or cellulose, or administered in an aerosol spray.

31. A method according to claim 23, wherein the lipid/s is formed into liposomes by known techniques, or mixed with oily or viscous preparations to form liposomes, or otherwise incorporated into or onto carrier particles of appropriately sized particulate material, such as colloidal silica, silicilic acid, kaolin particles or other such material capable of absorbing or adsorbing lipids.

32. A method according to claim 31, wherein the particles are incorporated into powder, tablets, capsules, food, liquids or beverages.

33. A method according to claim 31 or claim 32, wherein the liposomes or carrier particles incorporating the lipid/s have a size range of 0.1 to 100 microns, preferably 0.2 to 25 microns, most preferably 0.2 to 15 or even 0.3 to 10 microns, and ideally 0.4 to 8 microns or 0.5 to 5 microns.
